(19) United States
(12) Patent Application Publication
(10) Pub. No.: US 2004/0234551 A1
(43) Pub. Date: Nov. 25, 2004

(54) Use of 3'-utr's from cysteine proteinase genes cpb2 and cpb2.8 of leishmania for directing stage-specific expression

(76) Inventors: Graham Herbert Coomes, Glasgow (GB); Jeremy Charles Mottram, Glasgow (GB); Darren Robert Brooks, West Yorkshire (GB); Hubert Alexandre Denise, Glasgow (GB)

Correspondence Address: MYERS BIGEL SIBLEY & SAJOVEC, PO BOX 37428, RALEIGH, NC, 27627, US

(21) Appl. No.: 10/257,076

(22) PCT Filed: Apr. 12, 2001
(86) PCT No.: PCT/GB01/01664

(30) Foreign Application Priority Data
Apr. 12, 2000 (GB) 0008903.7

(51) Int. Cl. A61K 39/295; C12N 15/74; C12Q 1/68

(52) U.S. Cl. 424/201.1; 435/471

(57) ABSTRACT

The present invention relates to the identification and use of sequence elements present in the intergenic region of Cysteine Proteinase Genes, cpb, in L. mexicana and the observation that the identified sequences are involved in the control of stage-regulated gene expression. Principal uses include the preparation of vaccines.

### FIELD OF INVENTION

The present invention relates to the identification and use of sequence elements present in the intergenic region of Cysteine Protein Genes, cpb, in L. mexicana and the observation that the identified sequences are involved in the control of stage-regulated gene expression. Principal uses include the preparation of vaccines.

### BACKGROUND OF INVENTION

Protozoan parasites of the genus Leishmania are responsible for a spectrum of diseases, termed leishmaniasis, that afflict approximately 12 million individuals in tropical and sub-tropical regions. Infections range in severity from spontaneously healing cutaneous ulcers to potentially fatal visceral disease (kala azar). Leishmaniasis is also an important diseases of dogs. Antimonials (eg. pentostam) and diamidines (eg. pentamidine) though far from ideal, remain one of the few useful forms of leishmanial chemotherapy. The complex nature of the immune response to the various forms of infection has hindered progress towards a vaccine, though this still remains a priority. The parasite has a digenetic life cycle, passing between sandfly vector and mammalian hosts (dogs and rodents may act as reservoirs for human infections). Leishmania exist as extracellular flagellated promastigotes within the insect alimentary canal and these differentiate into the highly infectious metacyclic form that are responsible for transmission to mammals. Parasites are transmitted during a vector bloodmeal, and following macrophage invasion they reside within phagolysosomes as amastigotes.

Central to these developmental changes is variation in gene expression. Gene expression in Leishmania and other trypanosomatid differs in several important ways from that of most eukaryotes. Trypanosomatid protein coding genes generally lack promoter elements, they are closely spaced, often being found in tandem arrays encoding the same (or similar) open reading frames interspersed with unrelated sequences [1], and they have an absolute requirement for trans-splicing [2,3]. Most trypanosomatid genes are therefore transcribed polycistronically and regulation occurs predominantly at the post-transcriptional level [4]. Numerous studies [3,5 and references therein] have indicated that differential gene expression can be mediated by mRNA stability and other work [6] has shown that translational efficiency is also of importance. Both mechanisms are thought to be mediated by the 3′-untranslated regions of the respective genes [3,4,5]. Intergenic regions have also been implicated in controlling gene expression, presumably by mediating the events involved in pre-mRNA processing (trans-splicing and cleavage/polyadenylation) [7,8]. However, the precise mechanisms that govern trypanosomatid gene expression are not well understood.

The study of Leishmania gene regulation, especially during promastigote—amastigote differentiation, is important to the elucidation of the genes responsible for the transition to the parasite form that occurs in mammals and the molecular mechanisms underlying such changes.

U.S. Pat. No. 5,733,778 discloses sequence data from a differentially expressed gene, A2, from Leishmania donovani and its protein product, which is expressed at significantly higher levels in the amastigote (mammalian) stage of Leishmania. The A2 protein is recognised by kala-azar convalescent serum; kala azar, also know as visceral leishmaniasis, being the human disease caused by the species L. donovani. Mutants lacking expression of this gene may be potentially useful for the development of an attenuated strain of Leishmania, which cannot survive in humans but generates a protective immune response.

Other species of Leishmania contain differentially expressed genes. Leishmania mexicana, for example, contain a number of cathepsin L-like lysosomal cysteine proteinases (CPs) [9]. These Type I enzymes are expressed in increasing amounts during life cycle progression [10], such that in amastigotes the CPs represent approximately 1% of the total cell protein. The enzymes are encoded by the cpb genes, which map to one genomic locus as a tandem array of 19 copies [11]. Targeted deletion of this array has shown that the genes encode virulence factors [12]. Re-expression of different gene copies in the cpb null mutant and the subsequent analysis of enzyme substrate preferences have suggested that CPBs possess different substrate specificities [11]. The first two genes of the array (cpb1 and cpb2) are atypical since they encode enzymes that lack the C-terminal domain characteristic of trypanosomatid Type I cycsteine proteinases. Furthermore, Northern blotting has shown that cpb1 and cpb2 are expressed almost exclusively in the infective metacyclic stage; the ratio of mRNA between promastigote:metacyclic:amastigote as assessed by phosphorimage analysis is 1:6.5:0.2. This is in contrast to the remaining isogenes that are expressed predominantly in amastigotes (1:6:33) [11]. However, the mechanism that controls such stage-regulated gene expression in Leishmania is not well understood.

Generally speaking the present invention relates in part to the characterisation of a non-coding region that differs significantly between the metacyclic-specific cpb1 and cpb2 repeat units and the amastigote-specific gene, cpb2.8, and the observation that sequences from this region may be used to control protein expression in a stage-specific manner.

### SUMMARY OF INVENTION

In a first aspect the present invention provides a nucleic acid construct for use in stage-regulated expression of a polypeptide in Leishmania; the construct comprising:

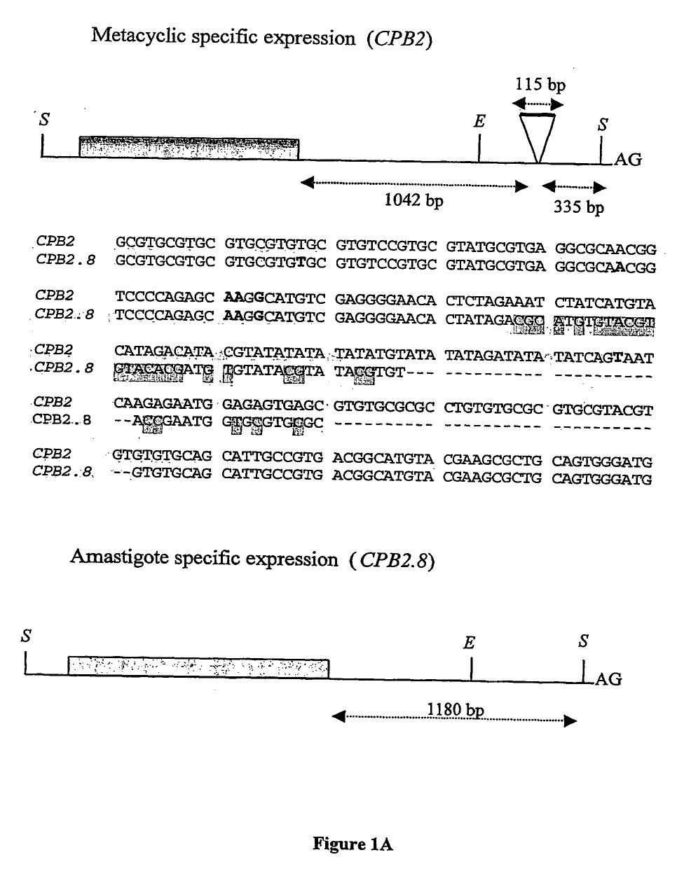

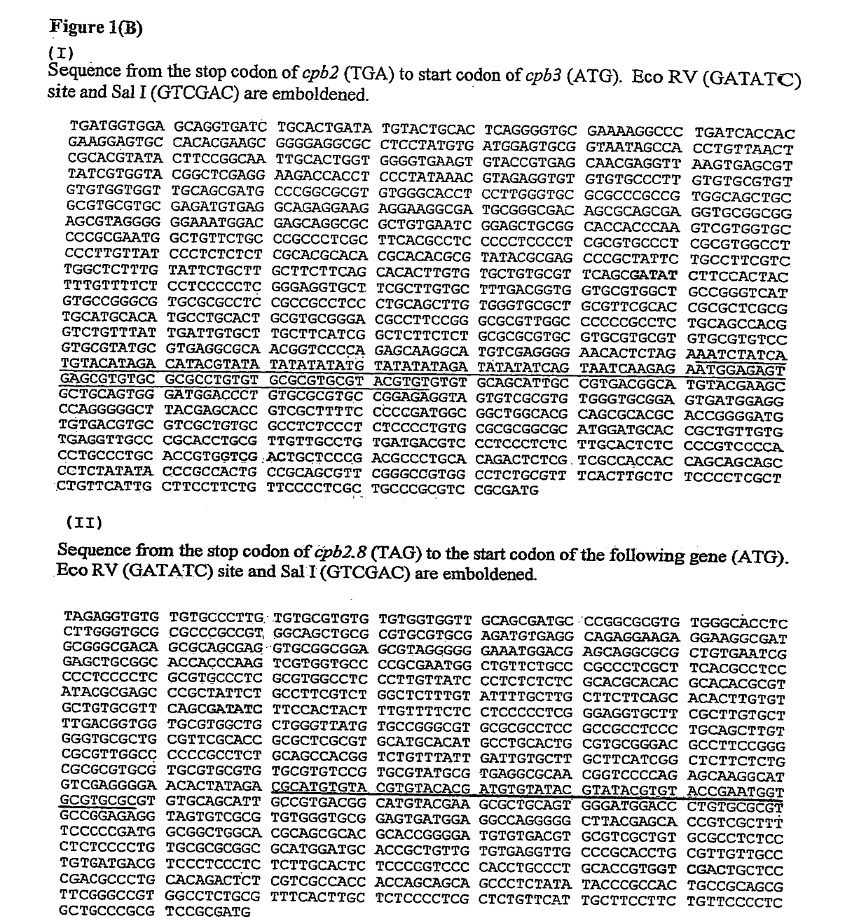

a first nucleic acid sequence comprising a stage-regulated control sequence substantially as shown in FIG. 1B(I) or (II), portion thereof or functional homologue thereof; and

a second or further nucleic acid sequence or sequences, operatively linked to said first nucleic acid sequence, encoding said polypeptide.

It should be understood that “functional homologue” relates to nucleic acid sequences with a similar function. That is, nucleic acid sequences capable of effecting said stage-regulated expression. Generally speaking, “functional homologue” relates to nucleic acid sequences from Leishmania sharing at least 25%, 50%, particularly 60, 70 and 80%, and especially 90 and 95% identity to the nucleic acid sequences shown in FIG. 1B(I) or (II) or portion thereof, for example, the nucleic acid sequence located between the EcoRV and SalI sites as shown in bold in FIG. 1B(I) or (II) or the nucleic acid sequences shown in FIG. 1A or the 115 bp insertion sequence underlined in FIG. 1B(I). % sequence identity may be determined when the alignment or comparison is conducted by a computer homology program or search algorithm known in the art. By way of example and not limitation, useful computer homology programs include the following: Basic Local Alignment Search Tool (BLAST) (www.ncbi.nlm.nih.gov) [16, 17] a heuristic search algorithm tailored to searching for sequence similarity which ascribes significance using the statistical methods of Karlin and Altschul [18, 19]. Five specific BLAST programs perform the following tasks:

1) The BLASTP program compares an amino acid query sequence against a protein sequence database.

2) The BLASTN program compares a nucleotide query sequence against a nucleotide sequence database.

3) The BLASTX program compares the six-frame conceptual translation products of a nucleotide query sequence (both strands) against a protein sequence database.

4) The TBLASTN program compares a protein query sequence against a nucleotide sequence database translated in all six reading frames (both strands).

5) The TBLASTX program compares the six-frame translations of a nucleotide query sequence against the six-frame translations of a nucleotide sequence database.

Smith-Waterman (database: European Bioinformatics Institute www.ebi.ac.uk/bic_sw/) [20] is a mathematically rigorous algorithm for sequence alignments.

FASTA (see [21] is a heuristic approximation to the Smith-Waterman algorithm. For a general discussion of the procedure and benefits of the BLAST, Smith-Waterman and FASTA algorithms see [22] and references cited therein.

“Nucleic acid sequence” as used herein refers to a chain of nucleotides such as deoxyribose nucleic acid (DNA) sequences and transcription products thereof, such as RNA, and includes double and single-stranded DNA, and RNA sequences derived therefrom. “Nucleic acid constructs” thus refers to a product comprising a plurality of said nucleic acid sequences.

Typically, the nucleic acid construct comprises said nucleic acid sequences in the form of a first nucleic acid sequence or “control sequence” and a second nucleic acid sequence, wherein said “control sequence” is operably linked to said second nucleic acid sequence such that it is capable of effecting the stage-regulated expression of said second nucleic acid sequence and its polypeptide product. It is understood that the first and second nucleic acid sequences may be provided in any order. Preferably, however, the first nucleic acid sequence is located downstream or 3′ from the second nucleic acid sequence.

More typically, the nucleic acid construct comprises 5′ and 3′ flanking regions for integration via homologous recombination, for example, into a host genome. For example, these flanking regions may be unique cpb gene sequences, which allow integration or said nucleic acid construct into the cpb locus of Leishmania. Said nucleic acid construct further comprises spliced-leader (SL) and polyadenylation sequences needed for the formation of mature mRNA. Examples of sequence elements directing splicing and polyadenylation of cpb and sat genes include the sequence flanking the SalI site in the L. mexicana cpb intergenic region [11]. The 1.3 kb of dhfr-ts sequence is used to control the formation of mature mRNA encoding a selectable marker eg. streptothricin acetyl transferase (sat). Processing of 5′ and 3′ regions are not mutually exclusive [7].

In general, the term “polypeptide” refers to a chain or sequence of amino acids displaying an activity of interest and does not refer to a specific length of product as such. The polypeptide if required, can be modified in vivo and/or in vitro, for example for glycosylation, amidation, carboxylation, phosphorylation and/or post-translational cleavage, thus inter alia, peptides, oligopeptides, proteins and fusion proteins are encompassed thereby.

Said “control sequence” according to the present invention comprises the nucleic acid sequence as shown in FIG. 1B(I) or (II) or portion thereof which is able to control stage-specific expression of said second nucleic acid sequence. Optionally, the “control sequence” according to the present invention is obtainable from Cosmid pGL648, as deposited with the ECCC and assigned Accession No.______. Cosmid pGL648 contains a large genomic fragment from L. infantum JPC M5. The fragment contains, at least, the unique 5′ flanking region and the two first genes of the Cysteine Proteinase B gene array. The present invention also allows the identification of related “control sequences” from other species and thus also relates to functional homologues thereof of the sequences shown in FIG. 1B(I) or (II) or obtainable from Cosmid pGL648 as described above. That is, the sequences of the present invention may be used to identify and/or clone related sequences from other species.

When said control sequence is from L. mexicana preferably said control sequence comprises a nucleic acid sequence located between the EcoRV and SalI sites as shown in bold in FIG. 1B(I) or (II). More particularly, the present inventors have observed a divergence of sequence between the intergenic region of the cpb1 and cpb2 or cpb2 and cpb3 genes and the intergenic region downstream of cpb2.8 in L. mexicana. This region is shown underlined in FIGS. 1B(I) and (II) respectively. It can be seen that the 115 bp sequence as shown in FIG. 1B(I) comprises an “insertion element” totalling 57 bp compared with the 58 bp sequence from the equivalent region as shown in FIG. 1B(II). This insertion site lies immediately downstream of the polyA addition sites of the cpb1 and cpb2 genes (as mapped by Reverse Transcriptase PCR) and is characterised by a 25 bp AT-rich insertion and a 32-bp GT-rich insertion, specific to the cpb1 and cpb2 intergenic regions. This insertion element is thought to be absent from the remaining repeats of the cpb gene tandem array and particularly, it is absent from the 3′ non-coding region of the cpb2.8 gene, as shown in FIG. 1B(II). Thus, in a preferred aspect the “control sequence” comprises the 115 bp or 58 bp region as shown underlined in FIGS. 1B(I) or (II) respectively.

As mentioned above, the observation and characterisation of the activity of the particularly defined sequences from the intergenic regions of the cysteine proteinase genes from L. mexicana allows selected sequences from other species, especially other Leishmania species, to be cloned. Thus, the control sequence according to the present invention also relates to species-specific variants of the nucleic acid sequences shown in FIG. 1B(I) or (II) or Cosmid pGL648 deposited with the ECCC under accession no.______ or sub-fragments as identified herein. The skilled man will readily understand that said L. mexicana nucleic acid sequences may be used to clone and use corresponding cpb intergenic regions in other Leishmania species including L. braziliensis, L. peruviana, L. guyanesis, L. major, L. amazonensis, L. infantum, L. chagasi and L. donovani.

It is to be understood that the nucleic acid constructs of the present invention provide “stage-regulated expression” of chosen polypeptides. That is, it is possible to control stage-specific expression of genes/polypeptides in any one stage of the Leishmania life cycle, particularly metacyclic-specific or amastigote-specific expression. Most particularly, expression of nucleic acid constructs comprising the “control sequence” as shown in FIG. 1B(I), comprising 115 bp sequence that includes the 57 bp “insertion element”, gives rise to metacyclic-specific gene expression. Alternatively, expression of nucleic acid constructs comprising the “control sequence” as shown in FIG. 1B(II), wherein the “insertion element” is absent, controls amastigote-specific expression.

Thus, such control sequences may be used to express stage-specific Leishmania genes and their polypeptide products in other stages of the Leishmania life cycle, to suppress expression of a gene normally expressed in metacyclic or amastigote stages, or to enable stage-regulated expression of a non-leishmanial gene and polypeptide product such as a second or further nucleic acid sequence in said nucleic acid construct according to the present invention. Thus, a method for high level expression of heterologous genes/polypeptides in the metacyclic or amastigote form of Leishmania is provided.

Typically, this second or further nucleic acid sequence is a Leishmania gene such as cystein proteinase, for example cpb gene or variant thereof e.g. cpb engineered to encode an inactive enzyme, or Leishmania gp63 or LACK genes or other immunogenic Leishmania genes. Alternatively, it is a non-leishmania gene such as a reporter gene (e.g. Chloroamphenicol Acetyl Transferase (CAT), Green Fluorescent Protein (CFP)) or a gene whose product may induce modulation of the host immune response (e.g. cytokine).

Thus, stage-regulated expression of such a reporter gene in said nucleic acid construct could be used to follow the long-term infection of Leishmania in vivo, or to distinguish between infected and non-infected host cells, such as mammalian host cells.

In a further aspect, the present invention provides the use of a nucleic acid construct according to the present invention for the stage-regulated expression of a gene in Leishmania, in the manufacture of a vaccine for the prophylaxis and/or treatment of Leishmaniasis. Specifically, it should be possible to express amastigote-specific genes in the metacyclic form and metacyclic/promastigote-specific genes in the amastigote form of the parasite. Parasites used could be wild type Leishmania or attenuated mutants (e.g.Δcpb, or cysteine proteinase-deficient mutant; PCT/GB99/00889). Thus, the host immune response will come into contact with leishmanial proteins not normally present in that life-cycle stage (and elicit a protective immunity). The nucleic acid construct may take the form of naked nucleic acid sequence, that is, a nucleic acid construct according to the present invention not bound up in a vector form, such as a plasmid form. The vaccine of the invention may optionally include a further nucleic acid construct expressing polypeptide(s) with an immunogenic function such as a cytokine. The further nucleic acid construct may be in the form of a further vector as described herein, for example an additional plasmid vector. Alternatively, the additional nucleic acid construct can be in the form of a naked DNA. Such naked DNA may be adhered to a microprojectile or in an appropriate holding solution, such as a saline solution. Alternatively, the nucleic acid construct can be available in the form of a vector or of a host cell.

In a preferred presentation, the vaccine can also comprise an adjuvant. Adjuvants in general comprise substances that boost the immune response of the host in a non-specific manner. A number of different adjuvants are known in the art. Examples of adjuvants may include Freund's Complete adjuvant, Freund's Incomplete adjuvant, liposomes, and niosomes as described in WO90/11092, mineral and non-mineral oil-based water-in-oil emulsion adjuvants, cytokines, short immunostimulatory polynucleotide sequences, for example in plasmid DNA containing CpG dinucleotides such as those described by Sato Y. et al. (1996); and Krieg A. M. (1996) [14, 15]. Further adjuvants of use in the invention include encapsulators comprising agents capable of forming microspheres (1-10 μm) such as poly(lactide-coglycolide), facilitating agents which are capable of interacting with polynucleotides such that the said polynucleotide is protected from degradation and which agents facilitate entry of polynucleotides such as DNA into cells. Suitable facilitating agents include cationic lipid vectors such as:

1,3-di-oleoyloxy-2-(6-carboxy-spermyl)-propylamid (DOSPER),

N-[1-(2,3-dioleoyloxy)propyl]-N,N,N-trimethylammoniummethylsulfate (DOTAP),

N-[1-(2,3-dioleoyloxy)propyl)]-N,N,N-trimethylammonium chloride (DOTMA),

(N,N,N′,N′-tetramethyl-N,N′-bis(2-hydroxylethyl)-2,3-dioleoyloxy-1,4-butanediammonium iodide, bupivacaine-HCl, non-ionic polyoxypropylene/polyoxyethylene block copolymers, polyvinyl polymers and the like.

Such cationic lipid vectors can be combined with further agents such as L-dioleoyl phosphatidyl ethanolamine (DOPE) to form multilamellar vesicles such as liposomes.

The mode of administration of the vaccine of the invention may be by any suitable route that delivers a suitable amount of the nucleic acid construct, or vector of the invention to the subject. However, the vaccine is preferably administered parenterally via the intramuscular or deep subcutaneous routes. Other modes of administration may also be employed, where desired, such as oral administration or via other parenteral routes, i.e., intradermally, intranasally, or intravenously.

Generally, the vaccine will usually be presented as a pharmaceutical formulation including a carrier or excipient, for example an injectable carrier such as saline or a pyrogenic water. The formulation may be prepared by conventional means.

It will be understood, however, that the specific dose level for any particular recipient animal will depend upon a variety of factors including age, general health, and sex; the time of administration; the route of administration; synergistic effects with any other drugs being administered; and the degree of protection being sought. Of course, the administration can be repeated at suitable intervals if necessary.

Typically, the nucleic acid construct may be provided in a vector such as a plasmid, virus or the like, suitable for introduction into a host cell, such as Leishmania, where such vectors may integrate into the host's genome or replicate autonomously in the particular cell.

Generally speaking, the nucleic acid constructs are expressed in host cells by use of a so-called expression vector. The skilled addressee will understand that such expression vectors contain sequences to promote and terminate the transcription/translation and correct processing of precursor mRNA to mature RNA and are well known in the art.

The nucleic acid construct and/or vector comprising the nucleic acid construct may be used for transformation of a suitable host. “Transformation”, as used herein, refers to the introduction of a heterologous polynucleotide fragment into a host cell, irrespective of the method used, for example direct uptake, electroporation transfection, transduction or the like.

Typically, although not exclusively, the host cells are of protozoan parasites, particularly of the genus Leishmania and the expression vector is one which is suitable for expression in the particular cell type. Species of Leishmania suitable as host cells for expression studies include L. mexicana, L. braziliensis, L. peruviana, L. guyanensis, L. major, L. amazonensis, L. infantum, L. chagasi and L. donovani. However, suitable hosts for use in cloning and maintaining the nucleic acid constructs and/or vectors comprising the nucleic acid constructs may be selected from bacteria, yeasts, insect cells and mammalian cells.

In a further aspect the present invention provides use of a stage-regulated control sequence as shown in FIG. 1B(I) or (II) or functional homologue thereof in modulating expression of genes in Leishmania. It should be appreciated that modulation of gene expression commonly refers to the suppression or silencing of gene expression. For example, to suppress the expression of genes in the promastigote, metacyclic or amastigote stages of the Leishmania life cycle, such that important molecules, such as virulence factors, are removed and an attenuated parasite is produced. Thus, by placing the metacyclic-specific “control sequence” downstream of an amastigote gene, the amastigote gene is silenced. Likewise, placing the amastigote-specific “control sequence” downstream of a metacyclic-specific gene the metacyclic gene is silenced. It is to be appreciated that the control sequence of the present invention could be part of a target validation programme by silencing specific genes in a stage-specific manner.

In a yet further aspect, the present invention provides the use of the stage-regulated control sequence according to the present invention to isolate proteins that bind to the control sequence and regulate stage-specific gene expression. These proteins may be useful as chemotherapeutic targets.

### DETAILED DESCRIPTION OF PREFERRED EMBODIMENTS

Embodiments of the present invention will now be described by way of example only, with reference to the Figures which show;

FIG. 1—(A) Diagrammatic representation of the L. mexicana sequence downstream of cpb2 and the sequence downstream of cpb2.8 highlighting the 57 bp of total insertion sequence specific to the cpb2 repeat unit, which lies within a 115 bp region that displays significant sequence variation between cpb2 and cpb2.8 repeat units. Highlighted are shown sequence differences; the locations of polyadenylation sites are underlined. The complete cpb2 and cpb2.8 non-coding sequences are also presented (B), with the 115 bp and 58 bp “control sequence” shown underlined in (I) and (II) respectively.

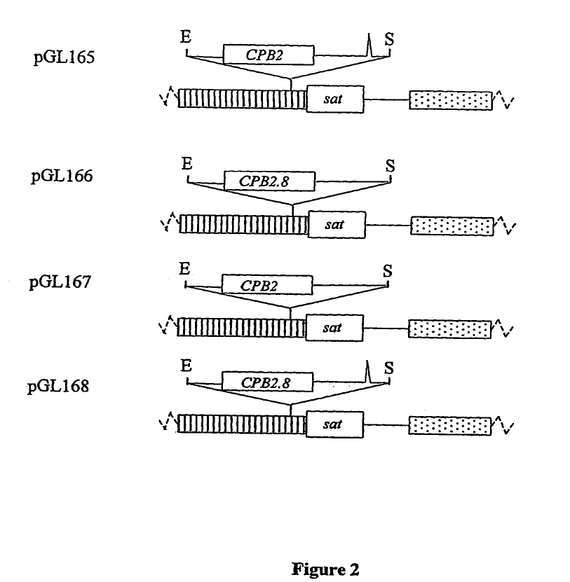

FIG. 2—Strategy utilised for the analysis of the capacity of the cpb non-coding regions to express stage-regulated CPBs. The L. mexicana null mutant was transfected with constructs encoding entire repeat units for cpb2 and cpb2.8. Stable integration of these constructs into the cpb locus of the null mutant was selected for by resistance to nourseothricin (conferred by sat gene) and concomitant loss of resistance to either hygromycin or phleomycin (marker genes used to generate Δ cpb null mutant)[12]. The common EcoRV and SalI sites (shown in bold in FIG. 1B) present in the non-coding regions of cpb2 and cpb2.8 were used to exchange the sequences of these two native repeat units. The resulting chimeric constructs were then used for transfection.

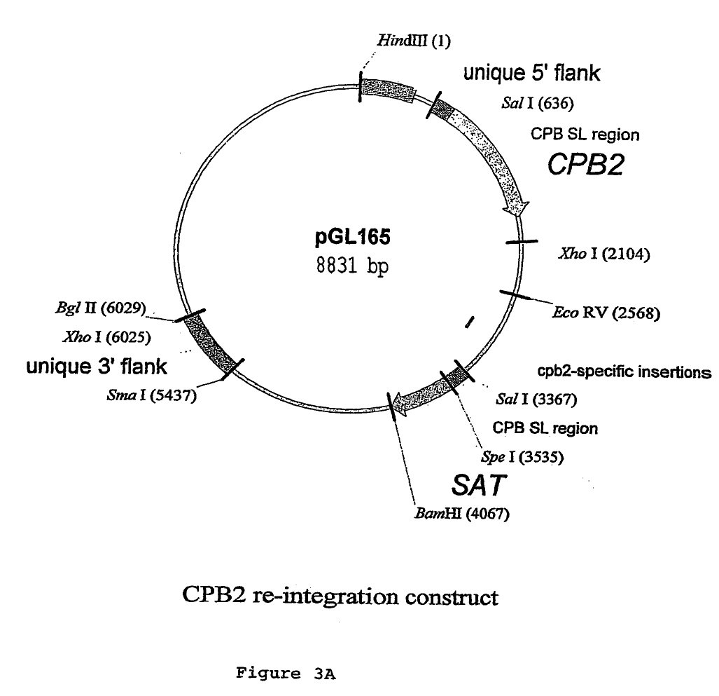

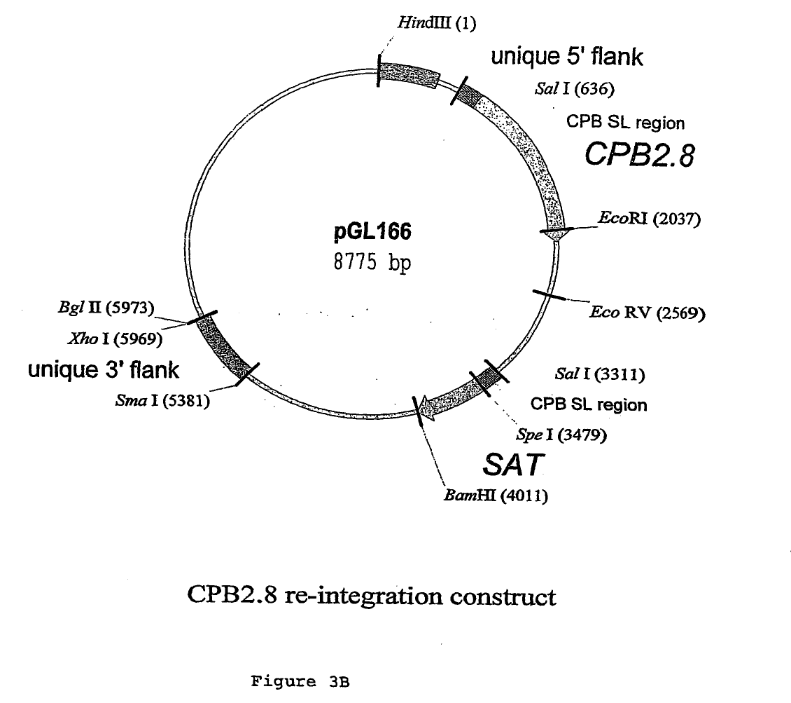

FIG. 3—Vector maps used in the strategy outlined in FIG. 2. Maps are shown for the native cpb2 re-integrant construct (A; also designated pGL165) and the native cpb2.8 re-integrant construct (B; also designated pGL166). Features common to both constructs include the unique cpb 5′ and 3′ flanking regions [11] cpb spliced-leader (SL) regions (from SalI site to start methionine of cpbs) needed for formation of mature mRNA for both the cpbs and selectable marker gene (sat) [11] immediately downstream of cpb ORFs is the non-coding region of cpb2/cpb2.8 and the cpb2-specific intergenic sequence is highlighted; immediately downstream of the sat ORF is approximately 1.3 kb of dhfr-ts sequence (derived from pX episome) [13] that is needed for formation of mature mRNA encoding the selectable marker streptothricin acetyl transferase (sat). The remainder of each plasmid (between 3′ and 5′ flanks) is sequence needed for maintenance and propagation in bacteria. The chimeric constructs (maps not shown) were generated by exchanging the non-coding EcoRV-SalI fragments of cpb2 and cpb2.8. For use in transfection of L. mexicana these plasmids are digested with HindIII and BglII to release the insertion cassette. The insertion cassette is purified (agarose gel electrophoresis, gel extraction, ethanol precipitation and washes) and resuspended in sterile water for transfection (requires 5-10 μg DNA) of late-log phase promastigotes. Selection of mutants is as described in FIG. 2.

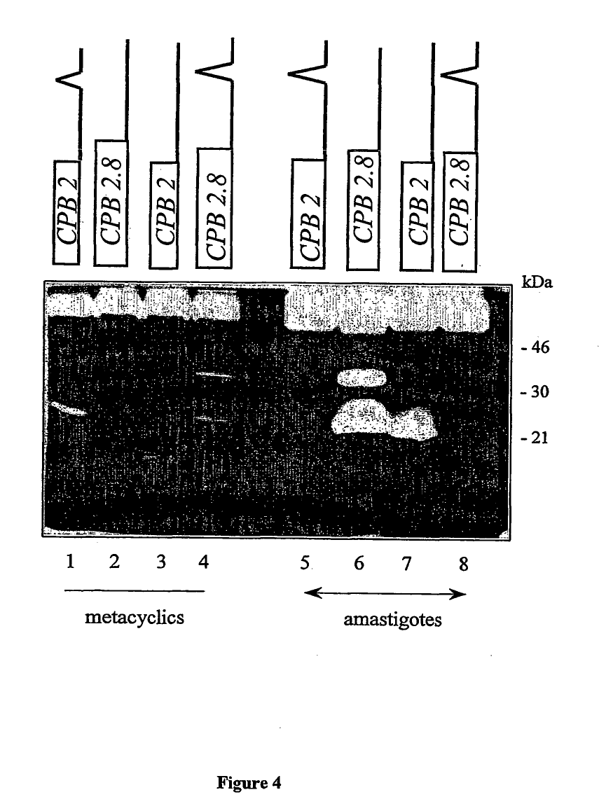

FIG. 4—Substrate gel (Gelatin-SDS-PAGE) analysis of the L. mexicana Δcpb null mutant re-expressing integrated native and chimeric constructs encoding CPB2 and CPB2.8. Briefly, 1×10⁷ metacyclic or amastigote cells were harvested and CPB activity was analysed by hydrolytic capacity towards gelatin (incorporated at 0.2% (w/v) in a standard SDS-PAGE gel). Gel shows that native CPB2 is active in metacyclics (lane 1) and not in mastigotes (lane 5) and that native CPB2.8 is not active in metacyclics (lane 2) but active in amastigotes respectively (ie. as wild type expression). In contrast, chimeric CPB2 is active in amastigotes (lane 7) and not in metacyclics (lane 3) and chimeric CPB2.8 is active in metacyclics but not in amastigotes (lane 8) (ie. reversal of wild type expression profiles).

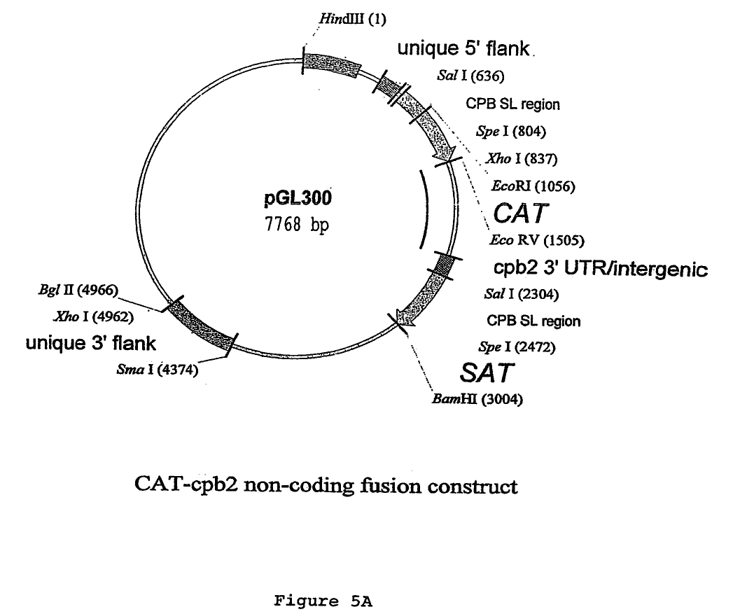

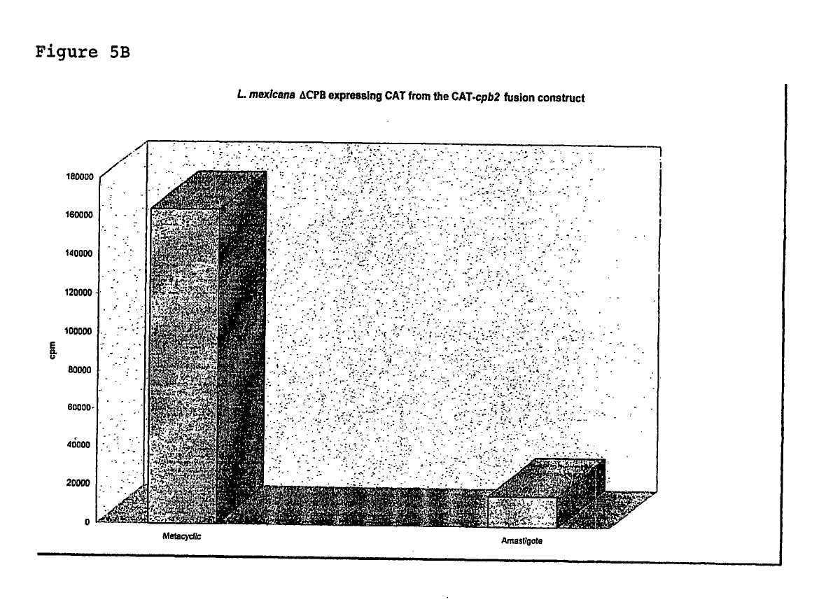

FIG. 5—Map of cat-cpb2 non-coding region fusion construct (A; also designated pGL300). The cat gene has been fused upstream of the cpb2 EcoRV-SalI non-coding region (includes the cpb2-specific insertion element) and then subcloned into the cpb re-integration plasmids (essentially as shown in FIG. 3). CAT reporter gene assay (B) of L. mexicana Δcpb expressing chloramphenicol acetyltransferase from the cat-cpb2 non-coding region fusion construct integrated at the cpb locus of the null mutant. Briefly, 1×10⁷ metacyclics or amastigotes were lysed and incubated with ¹⁴C-chloramphenicol and n-butyryl coenzyme A. The formation of n-butyryl chloramphenicol was then monitored by liquid scintillation counting. Expression of CAT is 1 order of magnitude higher in mteacyclics compared to amastigotes, confirming that the stage-specific expression of a heterologous protein is possible using L. mexicana non-coding gene regulatory elements.

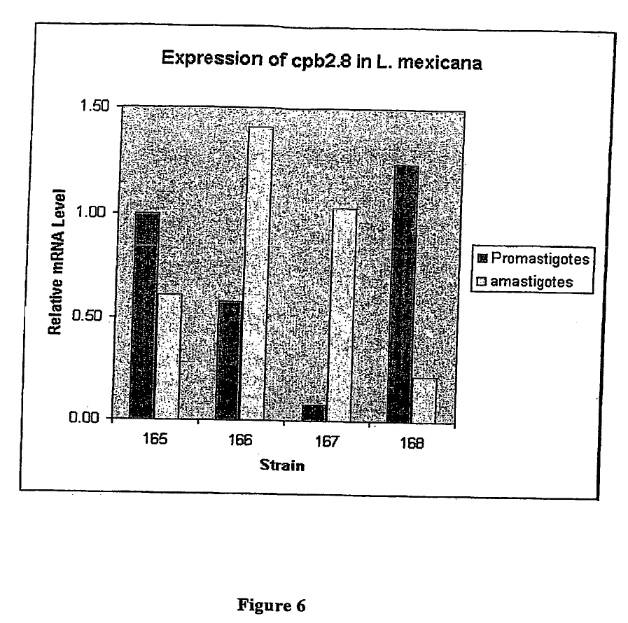

FIG. 6—Northern analysis of the RNA levels of CPB and mutant CPB genes re-integrated into the CPB locus of the CPB null mutant of Leishmania mexicana.

Key:

165—CPB2 with its native intergenic region

166—CPB2.8 with its native intergenic region

167—CPB2 with the CPB2.8 intergenic region

168—CPB2.8 with the CPB2 intergenic region

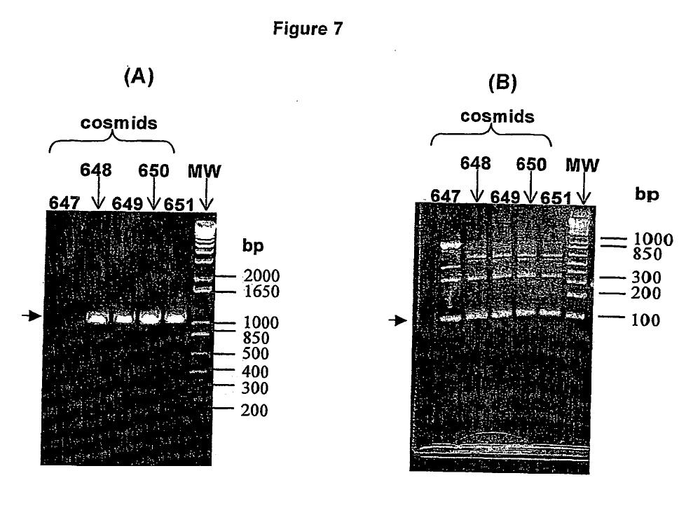

FIG. 7—Gel electrophoresis of PCR reactions amplifying CPB unique 5′ flanking regions of L. infantium cosmid

(A) OL661 and OL680 amplified at 1067 bp fragment corresponding to the unique 5′ flanking region of CPB on all the cosmid except pGL647.

(B) OL55 and OL191 amplified a 110 bp corresponding to the end of the non unique 5′ flanking region.

### EXAMPLE 1

Characteristics of 115 bp Non-Coding Region

Sequence analysis of Leishmania mexicana cpb genes identified a 115 bp sequence that differs in the non-coding region of these genes such that the metacyclic-specific cpb1 and cpb2 genes contains insertion elements that are not present in the amastigote-specific gene, cpb2.8 (FIG. 1B). This region is characterised by a 25 bp AT-rich insertion and a 32 bp GT-rich insertion, both of which are specific to the cpb1 and cpb2 intergenic regions located between cpb1-2 and cpb2-3 (FIG. 1A and FIG. 1B).

### EXAMPLE 2

Stable Integration of Native cpb2 and cpb2.8 Genes and Chimeric Versions of These Genes into Leishmania mexicana Genome and Expression Studies Therein

In order to determine whether stage-regulated cpb gene expression is controlled by sequence elements referred to in Example 1 above, a strategy was adopted that involves integration of the cpb repeat units, and variants thereof, back into the cpb locus of a L. mexicana cpb null mutant (FIG. 2). Cysteine proteinase activity was subsequently analysed for each mutant throughout the life-cycle (FIG. 3). As predicted, native repeat units, (cpb2 and cpb2.8) exhibited expression profiles analogous to that observed for the respective genes in wild-type cells. In contrast, chimeric versions of these genes (intergenic regions of cpb2 and cpb2.8 exchanged) exhibited a reversal of their expression profiles and hence confirmed the importance of the 115 bp cpb1/cpb2-specific intergenic region in the stage-regulation of CPBs.

### EXAMPLE 3

Stable Integration of the CAT Expression Construct into the Leishmania mexicana Genome and Expression Studies Therein.

The bacterial chloramphenicol acetyltransferase gene (cat) was used as a reporter gene in order to assess the relative capacities of a cpb non-coding region to control the stage-regulated expression of a heterologous gene. A construct was engineered such that the cat gene was fused to the cpb2 non-coding region (see FIG. 5A). This construct was targeted as before (FIG. 2) for integration at the cpb locus of the L. mexicana Δcpb null mutant. Data for the mutant expressing the cat-cpb2 non-coding construct show that expression is highly stage-specific, being an order of magnitude higher in metacyclic cells relative to that of amastigotes (FIG. 5B). We anticipate that L. mexicana mutants expressing the cat-cpb2.8 non-coding construct will also show a high degree of stage-regulation, with CAT activity this time being associated predominantly with amastigotes.

### EXAMPLE 4

Northern Analysis of the RNA Levels of CPB and Mutant CPB Genes Re-Integrated into the CPB Locus of the CPB Null Mutant of Leishmania mexicana.

Native genes and chimeric genes were integrated into the CPB locus of Leishmania mexicana from which the whole array of CPB genes had been deleted. The genes were either with their native 3′ intergenic region (CPB2 or CPB28) or with the 3′ intergenic region of the other gene. CPB2 is normally expressed primarily in metacyclic promastigotes and CPB2.8 is normally expressed primarily in amastigotes. The mutant parasites were then grown either as promastigotes or as axenic amastigotes and the CPB RNA levels determined using standard protocols. Relative mRNA levels were quantitated after hybridisation with an L. mexicana CPB probe using a Typhoon (Amersham) phosphoimager.

The results show that the presence of the CPB2 3′ intergenic region is correlated with RNA abundance primarily in promastigotes and the presence of the CPB2.8 intergenic region correlates with RNA abundance primarily in amastigotes (FIG. 6).

### EXAMPLE 5

[symbol] Generation of a Cosmid Library from Leishmania infantum JPC M5

Strain:

Leishmania infantum JPC clone M5 (MCAN/ES/98/LLM-877)

History:

the clone M5 was cloned by limited dilution from a log-phase promastigote culture of L. infantum strain JPC in RPMI.

L. infantum stock JPC (MCAN/ES/98/LLM-724) was isolated by spleen biopsy in NNN medium from a naturally infected dog. This strain was characterized as zymodeme 1 (MON-1).

Nature of the DNA:

genomic DNA

Vector Backbone:

SuperCos 1 cosmid vector (Stratagene, Amsterdam, The Netherlands).

Library Construction:

DNA isolation:

gDNA from L. infantum JPC clone M5 was isolated using Qiagen genomic-tip 20 kit for isolation of high-molecular weight DNA (Qiagen, Hilden, Germany).

DNA Preparation:

25 μg of gDNA was partially digested with Sau3AI (10′, 20′ and 30′ time points) and de-phosphorylated.

Vector Preparation:

25 μg of the SuperCos I was digested with XbaI, de-phosphorylated and digested with BamHI.

Ligation:

2.5 μg of partially digested genomic DNA

1 μg of SuperCost 1 digested vector 1 μl T4 DNA ligase (New England, Biolabs)

2 μl T4 DNA ligase buffer 10× water to final volume of 20 μl

Packaging:

Gigapack III XL packaging kit (Stratagene, Amsterdam, The Netherlands) was used according to the manufacturer's instructions.

The three packaging reactions (corresponding to the 10′, 20′ and 30′ time points were titered and pooled

The final titer of the library was 9.2×10⁸ cfu/ml (cfu: colony forming unit).

### EXAMPLE 6

[symbol] Isolation of CPB (Cysteine Proteinase B) Containing Cosmids

Probe:

1.5 kb PCR fragment containing the ORF of CPB gene from L. infantum JPCM5 clone.

Labelling:

The fragment was non-radioactively labeled with digoxigenin-11-dUTP (Boehringer Mannheim GmbH) in accordance with the manufacturer's instructions.

Screening:

Hybridizations were carried out under high-stringency conditions at 65° C. overnight.

After hybridization, the filters were washed at 65° C. two times 20 min. in 0.1x Sodium Citrate Solution (SSC) containing 0.1% SDS.

The immunological detection was carried out with anti-digoxigenin antibody conjugated to alkaline phosphatase.

The immune complexes were visualized by autoradiography after incubation 15 min at room temperature with the chemiluminescence substrate CSPF® (Boehringer Mannheim GmbH).

Five positive cosmid clones were isolated and named pGL647 to pGL651.

Cosmid pGL648 has been deposited in accordance with the Budapest Treaty with European Collection of Cell Cultures (Porton Down, Wiltshire, UK) on the ______ Apr. 2001 and is available under accession no.______. This deposit is characterised as follows:

Deposit Name: pGL648

Deposit Nature:

Recombinant DNA (cosmid) in the form of viable frozen cultures of bacteria.

Recombinant DNA:

Cosmid pGL648 containing a large genomic fragment from L. infantum JPC M5. The fragment contains, at least, the unique 5′ flanking region and the two first genes of the Cysteine Proteinase B gene array.

Cosmid Backbone:

SuperCosI (stratagene, Gebouw California, Hogehilweg 15, 1101 CB, Amsterdam Zuidoost, The Netherlands).

Bacteria Host:

XL1-Blue MR strain [D(mcrA)183 D(mcrCB-sdSMR-mrr) 173 endA1 supE44 thi-1 recA1 gyrA96-relA1 lac] (Stratagene).

Tube Content:

2 ml of bacteria culture in LB (Luria-Bertrani-medium) containing 20% glycerol and 1% peptone.

Culture Condition:

LB or other bacteria medium +50 μg/ml of ampicillin.

### EXAMPLE 7

[symbol] Amplification of CPB Unique 5′ Flanking Regions

Positions and Orientations of the Oligonucleotides:

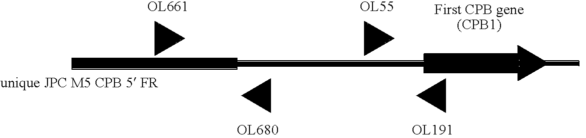

| Conditions of reaction: | 20 μl of 1X reaction PCR | |
|---|---|---|
| | buffer(Promega) with 1.5 mM | |
| | Mg²⁺; 100 μg/μl of each primer; | |
| | 50 ng of each cosmid and 1.25 | |
| | units of Taq (Promega) | |
| Conditions of amplification: | 1 cycle: | 95° C. 2 min |
| | 30 cycles: | 95° C. 10 s |
| | | 55° C. 30 s |
| | | 72° C. 1 min |
| | 1 cycle: | 72° C. 10 min |

Results:

OL661 and OL680 amplified a 1067 bp corresponding to the unique 5′ FR of CPB on all the cosmid except pGL647 FIG. 7 (A).

OL55 and OL191 amplified a 110 bp corresponding to the end of the non unique 5′FR FIG. 7(B).

### REFERENCES

[1] Myler, P. J, Audleman, L., DeVos, T., Hixson, G., Kiser, P., Lemley, C., Magness, C., Rickel, E., Sisk, E., Sunkin, S., Swartzell, S., Westlake, T., Bastien, P., Fu, G. L., Ivens, A. and Stuart, K. (1999) Leishmania major Friedlin chromosome 1 has an unusual distribution of protein-coding genes. Proc. Natl. Acad. Sci. USA 96, 2902-2906.

[2] Agabian, N. (1990) Trans-splicing of nuclear pre-messenger-RNAs. Cell, 61, 1157-1160.

[3] Vanhamme, L. and Pays, E. (1995) Control of gene-expression in trypanosomes. Microbiol. Rev. 59, 223-240.

[4] Graham, S. V. (1995) Mechanisms of stage-regulated gene-expression in kinetoplastida. Parasitol. Today, 11, 217-223

[5] Hotz, H. R., Biebinger, S., Flaspohler, J. and Clayton, C. (1998) PARP gene expression: control at many levels. Mol. Biochem. Parasitol. 91, 131-143.

[6] Hotz, H. R., Hartman, C., Huober, K., Hug, M. and Clayton, C. (1997) Mechanisms of developmental regulation in Trypanosoma brucei: a polypyrimidine tract in the 3 [symbol] -untranslated region of surface protein mRNA affects RNA abundance and translation. Nuc. Acids Res. 25, 3017-3025.

[7] Lebowitz, J. H., Smith, H. Q., Rusche, L. and Beverley, S. M. (1993) Coupling of poly(A) site selection and trans-splicing in Leishmania. Genes & Dev. 7, 996-1007

[8] Ramamoorthy, R., Swihart, K. G., McCoy, J. J., Wilson, M. E., and Donelson, J. E. (1995) Intergenic regions between tandem gp63 genes influence the differential expression of gp63 RNAs in Leishmania-chagasi promastigotes. J. Biol. Chem. 270, 12133-11139.

[9] Mottram, J. C., Brooks, D. R. and Coombs, G. H. (1998) Roles of cysteine proteinases of trypanosomes and Leishmania in host-parasite interactions. Curr. Opin. Microbiol. 1, 455-460.

[10] Robertson, C. D. and Coombs, G. H. (1994) Multiple high-activity cysteine proteases of Leishmania-mexicana are encoded by the lmcpb gene array. Microbiol. 140, 417-424.

[11] Mottram, J. C., Frame, M. J., Brooks, D. R., Tetley, L., Hutchison, J. E., Souza, A. E. and Coombs, G. H. (1997) The multiple cpb cysteine proteinase genes of Leishmania mexicana encode isoenzymes that differ in their stage regulation and substrate preferences. J. Biol. Chem. 272, 14285-14293.

[12] Mottram, J. C., Souza; A. E., Hutchison, J. E., Carter, R., Frame, M. J. and Coombs, G. H. (1996) Evidence from disruption of the lmcpb gene array of Leishmania mexicana that cysteine proteinases are virulence factors. Proc. Natl. Acad. Sci. USA 93, 6008-6013.

[13] Lebowitz, J. H., Coburn, C. M., McMahonPratt, D. and Beverley, S. M. (1990) Development of a stable Leishmania expression vector and application to the study of parasite surface-antigen genes. Proc. Natl. Acad. Sci. USA. 87, 9736-9740.

[14] Sato, Y. et al., (1996). Immunostimulatory DNA sequences necessary for effective intradermal gene immunization. Science 273, 352-354.

[15] Krieg, A. M., (1996). Trends in Microbiology, 4, 73-77

[16] Altschul et al., (1990). The BLAST Algorithm. J. Mol. Biol., 215, 403-410.

[17] Altschul et al., (1997). Nuc. Acids. Res., 25, 3389-3402.

[18] Karlin and Altschul, (1990). Proc. Natl. Acad Sci. USA, 87, 2264-2268.

[19] Karlin and Altschul, (1993). Proc. Natl/Acad. Sci. USA, 90, 5387-5877.

[20] Smith-Waterman, (1981). J. Mol. Biol., 147, 195-197.

[21] Pearson et al., (1988). Proc. Natl. Acad. Sci. USA, 85, 2444-2448.

[22] Nicholas et al., (1998). A tutorial on searching sequence databases and sequence scoring methods. www.psc.edu.

1-40. cancel.

41. A nucleic acid construct for use in stage-regulated expression of a polypeptide or polypeptides in Leishmania comprising:
a first nucleic acid sequence wherein said first nucleic acid is a stage-regulated control sequence consisting essentially of the sequence as shown in FIG. 1B(I) or (II), species-specific variants thereof, a functional portion thereof or functional homologue thereof; and
a second or further nucleic acid sequence or sequences, operatively linked to said first nucleic acid sequence, encoding said polypeptide or polypeptides.

42. A nucleic acid construct according to claim 41 further comprising 5′ and 3′ flanking regions for integration into a Leishmania host genome.

43. A nucleic acid construct according to claim 42, wherein the flanking regions are cpb sequences for integration into the cpb locus of a Leishmania hos genome.

44. A nucleic acid construct according to claim 41 wherein the stage-regulated control sequence can control stage-specific expression of genes/polypeptides in any one stage of the Leishmania life cycle.

45. A nucleic acid construct according to claim 41 wherein the stage-specific expression is metacyclic-specific expression and said stage-regulated control sequence consists essentially of the sequence as shown in FIG. 1B (I), species-specific variant thereof, a functional portion thereof or functional homologue thereof.

46. A nucleic acid construct according to claim 41, wherein the stage-specific expression is amastigote-specific expression and said stage-regulated control sequence consists essentially of the sequence as shown in FIG. 1(B) (II), species-specific variant thereof, a functional portion thereof or functional homologue thereof.

47. A nucleic acid construct according to claim 45 wherein the control sequence comprises the nucleic acid sequence located between the EcoRV and SalI sites substantially as shown in FIG. 1B (I) or species-specific variants thereof.

48. A nucleic acid construct according to claim 46 wherein the control sequence comprises the nucleic acid sequence located between the EcoRV and SalI sites substantially as shown in FIG. 1B (II) or species-specific variants thereof.

49. A nucleic acid construct according to claim 45 wherein the control sequence comprises the 115 bp sequence as shown underlined in FIG. 1B(I) or species-specific variants thereof.

50. A nucleic acid construct according to claim 46 wherein the control sequence comprises the 58 bp sequence as shown underlined in FIG. 1B (II) or species-specific variants thereof.

51. A nucleic acid construct according to claim 41 wherein the control sequence is from L. braziliensis, L. peruviana, L. guyanensis, L. major, L. mazonensis, L. chagasi and/or L. donovani.

52. A nucleic acid construct according to claim 41 wherein the control sequence is from L. infantum.

53. A nucleic acid construct according to claim 41 wherein the control sequence is obtainable from Cosmid pGL648, as deposited with the ECCC and assigned accession number 1041201.

54. A nucleic acid construct according to claim 41 wherein the second nucleic acid sequence is a Leishmania gene.

55. A nucleic acid construct according to claim 41 wherein the second nucleic acid sequence is a cysteine proteinase.

56. A nucleic acid construct according to claim 41 wherein the second nucleic acid sequence is a Leishmania cpb gene or variant thereof.

57. A nucleic acid construct according to claim 56 wherein the variant is cpb gene engineered to encode an inactive enzyme, or Leishmania gp63, LACK or other immunogenic Leishmania genes.

58. A nucleic acid construct according to claim 41 wherein the second nucleic acid sequence is a non-Leishmania gene.

59. A nucleic acid construct according to claim 58 wherein the non-Leishmania gene is a reporter gene.

60. A nucleic acid construct according to claim 59 wherein the reporter gene is a Chloramphenicol Acetyl Transferase (CAT) gene or Green Fluorescent Protein (GFP).

61. A nucleic acid construct according to claim 41 wherein the second nucleic acid sequence is a gene whose-products may induce modulation of the host immune response.

62. A nucleic acid construct according to claim 61 wherein the gene is a cytokine.

63. A method of following the long term infection of Leishmania in vivo comprising the step of providing to a host a nucleic acid construct according to claim 41

64. A method of distinguishing between infected and non-infected host cells comprising the step of providing a nucleic acid construct according to claim 41.

65. A method of treating Leishmaniasis comprising providing to a mammalian host a nucleic acid construct according to claim 41 in an effective amount.

66. A method of treating Leishmaniasis according to claim 65 wherein the nucleic acid construct is a naked nucleic acid sequence.

67. A vaccine comprising a nucleic acid construct according to claim 41.

68. A vaccine according to claim 67 capable of expressing amastigote-specific genes in the metacyclic form of a parasite.

69. A vaccine according to claim 67 capable of expressing metacyclic/promastigote-specific genes in the amastigote form of a parasite.

70. A vaccine according to claim 68 wherein the parasite is a wild type Leishmania parasite or an attenuated mutant parasite.

71. A vaccine according to claim 70 wherein the attenuated mutant is Δ cpb, or cysteine-proteinase deficient mutant.

72. A vaccine according to claim 67 further comprising an adjuvant.

73. A vaccine according to claim 72 wherein the adjuvant is selected from Freund's Complete adjuvant, Freund's Incomplete adjuvant, liposomes, niosomes, mineral and non-mineral oil-based water-in-oil emulsion adjuvants, cytokines and short immunostimulatory polynucleotide sequences.

74. An isolated nucleotide fragment consisting essentially of the nucleic acid sequence substantially as shown in FIG. 1B (I) or (II).

75. An isolated nucleotide fragment consisting essentially of the nucleic acid sequence located between the EcoRV and SalI sites substantially as shown in FIG. 1B (I) or (II).

76. An isolated nucleotide fragment consisting essentially of the 115 bp sequence as shown underlined in FIG. 1B (I) or species-specific variants thereof.

77. An isolated nucleotide consisting essentially of the 58 bp sequences as shown underlined in FIG. 1B (II) or species-specific variant thereof.

78. An isolated nucleotide fragment according to claim 76 wherein said species-specific variant is from L. infantum.

79. An isolated nucleotide fragment according to claim 77 wherein said species-specific variant is from L. infantum.

80. An isolated nucleotide fragment according to claim 74 wherein the nucleic acid sequence is obtainable from cosmid pGL648, as deposited with the ECCC and assigned accession number 1041201.

81. A method of controlling stage regulated gene expression in Leishmania comprising the steps of providing to a host an isolated nucleotide fragment according to any of claim 74.


SEQUENCE LISTING (2 sequences)

SEQ ID NO: 1 - DNA, 1657 bp, Leishmania mexicana
  misc_feature (1042)..(1156): 115 bp “control sequence”
tgatggtggagcaggtgatctgcactgatatgtactgcactcaggggtgcgaaaaggccctgatcaccacgaaggagtgccacacgaagcggggaggcgcctcctatgtgatggagtgcggtaatagccacctgttaactcgcacgtatacttccggcaattgcactggtggggtgaagtgtaccgtgagcaacgaggttaagtgagcgttatcgtggtacggctcgaggaagaccacctccctataaacgtagaggtgtgtgtgcccttgtgtgcgtgtgtgtggtggttgcagcgatgcccggcgcgtgtgggcacctccttgggtgcgcgcccgccgtggcagctgcgcgtgcgtgcgagatgtgaggcagaggaagaggaaggcgatgcgggcgacagcgcagcgaggtgcggcggagcgtaggggggaaatggacgagcaggcgcgctgtgaatcggagctgcggcaccacccaagtcgtggtgccccgcgaatggctgttctgcccgccctcgcttcacgcctccccctcccctcgcgtgccctcgcgtggcctcccttgttatccctctctctcgcacgcacacgcacacgcgtatacgcgagcccgctattctgccttcgtctggctctttgtattctgcttgcttcttcagcacacttgtgtgctgtgcgttcagcgatatcttccactactttgttttctcctccccctcgggaggtgcttcgcttgtgctttgacggtggtgcgtggctgccgggtcatgtgccgggcgtgcgcgcctccgccgcctccctgcagcttgtgggtgcgctgcgttcgcaccgcgctcgcgtgcatgcacatgcctgcactgcgtgcgggacgccttccgggcgcgttggccccccgcctctgcagccacggtctgtttattgattgtgcttgcttcatcggctcttctctgcgcgcgtgcgtgcgtgcgtgtgcgtgtccgtgcgtatgcgtgaggcgcaacggtccccagagcaaggcatgtcgaggggaacactctagaaatctatcatgtacatagacatacgtatatatatatatgtatatatagatatatatcagtaatcaagagaatggagagtgagcgtgtgcgcgcctgtgtgcgcgtgcgtacgtgtgtgtgcagcattgccgtgacggcatgtacgaagcgctgcagtgggatggaccctgtgcgcgtgccggagaggtagtgtcgcgtgtgggtgcggagtgatggaggccagggggcttacgagcaccgtcgcttttcccccgatggcggctggcacgcagcgcacgcaccggggatgtgtgacgtgcgtcgctgtgcgcctctccctctcccctgtgcgcgcggcgcatggatgcaccgctgttgtgtgaggttgcccgcacctgcgttgttgcctgtgatgacgtccctccctctcttgcactctccccgtccccacctgccctgcaccgtggtcgactgctcccgacgccctgcacagactctcgtcgccaccaccagcagcagccctctatatacccgccactgccgcagcgttcgggccgtggcctctgcgtttcacttgctctcccctcgctctgttcattgcttccttctgttcccctcgctgcccgcgtccgcgatg

SEQ ID NO: 2 - DNA, 1349 bp, Leishmania mexicana
  misc_feature (791)..(848): 58 bp “control sequence”
tagaggtgtgtgtgcccttgtgtgcgtgtgtgtggtggttgcagcgatgcccggcgcgtgtgggcacctccttgggtgcgcgcccgccgtggcagctgcgcgtgcgtgcgagatgtgaggcagaggaagaggaaggcgatgcgggcgacagcgcagcgaggtgcggcggagcgtaggggggaaatggacgagcaggcgcgctgtgaatcggagctgcggcaccacccaagtcgtggtgccccgcgaatggctgttctgcccgccctcgcttcacgcctccccctcccctcgcgtgccctcgcgtggcctcccttgttatccctctctctcgcacgcacacgcacacgcgtatacgcgagcccgctattctgccttcgtctggctctttgtattttgcttgcttcttcagcacacttgtgtgctgtgcgttcagcgatatcttccactactttgttttctcctccccctcgggaggtgcttcgcttgtgctttgacggtggtgcgtggctgctgggttatgtgccgggcgtgcgcgcctccgccgcctccctgcagcttgtgggtgcgctgcgttcgcaccgcgctcgcgtgcatgcacatgcctgcactgcgtgcgggacgccttccgggcgcgttggccccccgcctctgcagccacggtctgtttattgattgtgcttgcttcatcggctcttctctgcgcgcgtgcgtgcgtgcgtgtgcgtgtccgtgcgtatgcgtgaggcgcaacggtccccagagcaaggcatgtcgaggggaacactatagacgcatgtgtacgtgtacacgatgtgtatacgtatacgtgtaccgaatggtgcgtgcgcgtgtgcagcattgccgtgacggcatgtacgaagcgctgcagtgggatggaccctgtgcgcgtgccggagaggtagtgtcgcgtgtgggtgcggagtgatggaggccagggggcttacgagcaccgtcgcttttcccccgatggcggctggcacgcagcgcacgcaccggggatgtgtgacgtgcgtcgctgtgcgcctctccctctcccctgtgcgcgcggcgcatggatgcaccgctgttgtgtgaggttgcccgcacctgcgttgttgcctgtgatgacgtccctccctctcttgcactctccccgtccccacctgccctgcaccgtggtcgactgctcccgacgccctgcacagactctcgtcgccaccaccagcagcagccctctatatacccgccactgccgcagcgttcgggccgtggcctctgcgtttcacttgctctcccctcgctctgttcattgcttccttctgttcccctcgctgcccgcgtccgcgatg